(19) United States
(12) Patent Application Publication
(10) Pub. No.: US 2019/0300866 A1
(43) Pub. Date: Oct. 3, 2019

(54) METHOD FOR CLONING AND EXPRESSION OF PFOI RESTRICTION ENDONUCLEASE

(71) Applicants: THERMO FISHER SCIENTIFIC BALTICS UAB, Vilnius (LT); VILNIUS UNIVERSITY, Vilnius (LT)

(72) Inventors: Sonata JURENAITE-URBANAVICIENE, Vilnius (LT); Gintautas TAMULAITIS, Vilnius (LT)

(21) Appl. No.: 16/369,641

(22) Filed: Mar. 29, 2019

Related U.S. Application Data
(60) Provisional application No. 62/650,493, filed on Mar. 30, 2018.

(51) Int. Cl. C12N 9/16

(52) U.S. Cl. CPC C12N 9/16

(57) ABSTRACT

This application relates to the isolated DNA encoding the PfoI restriction endonuclease, vectors containing the isolated DNA and host cells expressing the vectors as well as a method for producing recombinant PfoI restriction endonuclease.

### CROSS-REFERENCE

This application claims the benefit under 35 U.S.C. § 119(e) of U.S. Provisional Application No. 62/650,493, filed 30 Mar. 2018, the entire contents of which are incorporated by reference herein.

### REFERENCE TO A SEQUENCE LISTING

The instant application contains a Sequence Listing which has been submitted electronically in ASCII format and is hereby incorporated by reference in its entirety. Said ASCII copy, created on Mar. 5, 2019, is named LT01337_ST25_SubstSL.txt and is 18,198 bytes in size.

### FIELD

Recombinant endonucleases, as well as methods for preparing and using them.

### BACKGROUND

Restriction endonucleases are enzymes that occur naturally mostly in bacteria and archaea and that function to protect these organisms from infections by viruses and other destructive DNA elements. Restriction endonucleases bind to specific nucleotide (nt) sequences in double-stranded DNA molecules (dsDNA) and cleave the DNA, often within or close to these sequences, fragmenting the molecules and triggering their degradation. Restriction endonucleases commonly occur with one or more accompanying modification enzymes, known as methyltransferases. The methyltransferases bind to the same nt sequence in dsDNA as the restriction endonuclease, but instead of cleaving the DNA, they methylate one of the bases in each strand of the sequence. This modification prevents the restriction endonuclease from binding to that site, thereby making the site resistant to cleavage. Methyltransferases act as cellular remedy to the restriction endonucleases they accompany, protecting the DNA of the cell from destruction by its own restriction endonucleases. The restriction endonuclease can recognize and cleave the foreign DNA because it is not methylated, whereas the DNA of the host is protected from cleavage due to the action of a host methyltransferase. Restriction systems allow bacteria to monitor the origin of incoming DNA and to destroy it, if it is recognized as foreign.

Together, a restriction endonuclease and its “cognate” methyltransferase(s) form a restriction-modification (R-M) system. The major biological function of the restriction-modification system is to protect the host from bacteriophage infection (Arber, Science, 205:361-365 (1979)). Other functions have also been suggested, such as involvement in recombination and transposition (Carlson, et al., Mol Microbiol, 27:671-676 (1998); Heitman, Genet EngQAY), 15:57-108 (1993); McKane, et al., Genetics, 139:35-43 (1995)).

A large and varied group of restriction endonucleases termed “Type II” cleave DNA at defined positions, and can be used in the laboratory to cut DNA molecules into precise fragments for gene cloning and analysis. The biochemical precision of type II restriction endonucleases exceeds anything achievable by chemical methods, therefore these endonucleases have become the main tools of modern molecular biology. They are the “scissors” by means of which genetic engineering and analysis is performed, and their adoption has profoundly impacted the biomedical sciences over the past 35 years. Their usefulness has triggered a continuous search for new restriction endonucleases, and a large number have been found. Today more than 200 Type II endonucleases possessing different DNA cleavage characteristics are known (Roberts et al, Nucl. Acids Res. 38: D234-D236 (2010)); (REBASE®, rebase.neb.com/rebase). Restriction endonucleases can be obtained by isolating them from the host bacteria and/or archaea that produce them, but these microorganisms can be difficult to culture, and expression levels may be lower than desired. Thus, there is a need in the art for improved production and purification of restriction endonucleases by the cloning and over-expression of the genes that encode them in non-natural production strain host cells such as E. coli that could potentially offer culture and expression-level advantages.

Since various restriction endonucleases perform similar roles in nature, and do so in much the same ways, it might be thought that they would resemble one another closely in amino acid sequence, organization, and behavior. Surprisingly, far from resembling one another, most Type II restriction endonucleases appear unique, resembling neither other restriction endonucleases nor any other known kind of protein. Type II restriction endonucleases seem to have evolved independently of one another, so that today's endonucleases represent a heterogeneous collection rather than a discrete family. The orders and orientations of restriction and modification genes vary, with all possible organizations occurring. Several kinds of methyltransferases exist, some methylating adenines, others methylating cytosines at the N-4 position, or at the C-5 position. Usually there is no way of predicting, a priori, which modifications will block a particular restriction endonuclease, which kind(s) of methyltransferases(s) will accompany that restriction endonuclease in any specific instance, nor what their gene orders or orientations will be.

From the point of view of cloning a Type II restriction endonuclease, the great variability that exists among restriction-modification systems means that, for experimental purposes, each endonuclease is unique in its amino acid sequence and catalytic behavior; each occurs in unique enzymatic association, adapted to unique microbial circumstances; and each presents the experimenter with a unique challenge. Sometimes a restriction endonuclease can be cloned and over-expressed in a straightforward manner but more often it cannot, and what works well for one endonuclease would not work at all for the next. Success with one is no guarantee of success with another.

### SUMMARY

In accordance with the description, this application describes recombinant PfoI restriction endonuclease. This application also describes recombinant vectors comprising the nucleic acid sequence of PfoI, host cells expressing recombinant PfoI, and methods of expressing PfoI.

In some embodiments, a recombinant vector comprises a recombinant DNA of SEQ ID NO: 1.

In some embodiments, a recombinant vector comprises a nucleic acid that encodes a restriction endonuclease polypeptide with at least 90% sequence identity to SEQ ID NO: 2. In some embodiments, the recombinant vector further comprises at least one regulatory element from a different organism than the DNA of SEQ ID NO: 1 or the polypeptide of SEQ ID NO: 2.

In some embodiments, the regulatory element comprises at least one of a promoter, a translation initiation sequence, a termination codon, a transcription termination sequence, or a sequence encoding a protein tag.

In some embodiments, the invention comprises host cell transformed by a recombinant vector. In some embodiments, the host cell comprises bacterial expression cells, yeast expression cells, algae expression cells, insect expression cells, mammalian expression cells, and cell-free in vitro expression systems.

In some embodiments, the invention comprises a method of producing recombinant PfoI restriction endonuclease from a host cell. In some embodiments, the method comprises premodifying the DNA sequence TCCNGGA in the host cell by methylation at one or more nucleotides and culturing the premodified host cell under conditions for expression of PfoI restriction endonuclease. In some embodiments, premodifying the DNA in the host cell is achieved by transforming the host cell with a vector comprising the recombinant DNA of SEQ ID NO: 8 and causing methylation of the host cell DNA by culturing the host cell under conditions for expression of M.Ec118kI.

In some embodiments, the invention comprises a method of producing a restriction endonuclease, comprising culturing a host cell under conditions suitable for expression of the restriction endonuclease. In some embodiments, the host cell is also transformed with a vector comprising the recombinant DNA encoding a methyltransferase capable to modify one or more nucleotides of the host cell DNA sequence TCCNGGA. In some embodiments, the host cell is transformed with a vector comprising the recombinant DNA of SEQ ID NO: 8.

In some embodiments, the restriction endonuclease cleaves DNA at the nucleotide sequence TCCNGGA.

In some embodiments, the invention comprises a host cell transformed with a recombinant vector comprising a nucleic acid of SEQ ID NO: 1, and a restriction endonuclease encoded by SEQ ID NO: 1 binds to the nucleotide sequence TCCNGGA and cleaves between T and C in each strand, producing DNA fragments with protruding pentanucleic 5′-ends.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1A and FIG. 1B shows native and recombinant R.Pfo activity in cell-free extracts. FIG. 1A shows lambda DNA digested for 10 minutes at 37° C. by 1, 2 or 5μ of 100 times diluted, 10 times diluted and undiluted cell-free extracts of Pseudomonas fluorescens biovar 126 and 1, 2 or 5 μl of 100 times and 10 times diluted cell-free extracts of E. coli BL21 (DE3)/pACYC_M.Ec118kI/pET21b(+)_R.PfoI, respectively. FIG. 1B shows lambda DNA digested for 1 hour at 37° C. by 1, 1.5, 2.0, 2.5, 3.0, 4.0, 5.0 μl of 50 times diluted cell-free extracts of Pseudomonas fluorescens biovar 126 and E. coli BL21 (DE3)/pACYC_M.Ec118kI/pET21b(+)_R.PfoI, respectively. M—GeneRuler DNA Ladder Mix (Thermo Scientific); K—lambda DNA digested with R.PfoI (Thermo Scientific); “-” denotes control undigested lambda DNA. Numbers above the picture indicate amount in μl of the diluted extracts added to the respective reaction mixtures.

FIG. 2 shows SDS-PAGE analysis of cell-free extracts. M—PageRuler Prestained Protein Ladder (Thermo Scientific); P—cell-free extract of Pseudomonas fluorescens biovar 126; U—E. coli BL21 (DE3)/pACYC_M.Ec118kI/pET21b(+)_R.PfoI cells incubated without IPTG induction; I—E. coli BL21 (DE3)/pACYC_M.Ec118kI/pET21b(+)_R.PfoI cells incubated after IPTG induction; K—70 units of PfoI (Cat. No. ER1751, Thermo Scientific). In K lane, BSA from PfoI storage is also seen (a protein band of higher molecular weight).

FIG. 3 shows alignment of amino acids 5-319 of hypothetical protein [Bacillus sp. Leaf13] (Subject; SEQ ID NO: 9) with amino acids 2-310 of R.PfoI (Query; SEQ ID NO: 2).

### DESCRIPTION OF THE SEQUENCES

Table 1 provides a listing of certain sequences referenced herein.

TABLE 1
Description of the Sequences

| | | SEQ |
|---|---|---|
| Descrip- | | ID |
| tion | Sequences | NO |
| R.PfoI | ATGCAGAAGTATAGGTTGTATGAGAAGGATGGCTCGCC | 1 |
| nucleic | TGTTCAAGACTTCAATCGATTTGTAAAAGGCTGGTTAG | |
| acid | ATATTGAGTTTGGTTTAAAGGAGCATCAGCCGCCAAAG | |
| sequence | GTATTTGATACTATAAGAGATAAGTATAACGAAGCTAT | |
| | AGAAGCTGTTGTATTGAGCGGTGTTGCGCCGCGGACGG | |
| | CACATAAGGCTGCGCTTTCCACTTTGACCGAACTTCTA | |
| | TTTGGTCATGATCTTGCAAAAGAACTTAGTGCTCGTTT | |
| | GGATATTCAGCCCATAGGAGTAGGCGGATTTCGTTCGG | |
| | CGCATTCTCAAGCATTTGCTAAAAATGTTGGCGAGAAC | |
| | TTTGTAAATTTAATGGTCTATGCGCTTGCGTGTATCTT | |
| | GAAAGATAATGATGATGTTCTGGTAGATAAGGGGCTGC | |
| | CACCTCATCTAAAAAAAGCTCTAACATTGAGTCGTGAG | |
| | TGTAGGATTAAAGATACGCTTCGTGAAATAAAGATCCC | |
| | TATAGAAGGGGATCTGTGTGTTTTTTCACGAAGTAATC | |
| | ATTGCAATGCAATCGTTATTAGCGCTAAAACAAGACTC | |
| | AAAGAAGTCTTTCATATTGGCACAATGTGGGCGTTGTT | |
| | TTCTGATGTGGCGAAAGATGAATACTGTCTTAACAAGT | |
| | GGGGGCTGAAAGTTGAGTCGTCAGAGTCCCTCAAGGAT | |
| | ACGATGTATGTATTTGCTACAGCTGATATGATAAATAA | |
| | AGATGGTGCAAGAAGTCAGGGTTGTGATGTGGAGAGGG | |
| | AAACTCCAAGAAACTTAATTGCGATGGACGCCTCTTTT | |
| | TTTGATTATGTTTTTGTTTCGAAGATGGGTATCGGGCA | |
| | TGTCTCTTCCGATCTTTCTCTTAAGTATGGGCGAGAAT | |
| | CGCTCTTCCACGAGCTAGGGTGCATAATTGATATGATC | |
| | GAACAAAAGTTTGATATTTTACTT | |
| R.PfoI | MQKYRLYEKDGSPVQDFNRFVKGWLDIEFGLKEHQPPK | 2 |
| amino | VFDTIRDKYNEAIEAVVLSGVAPRTAHKAALSTLTELL | |
| acid | FGHDLAKELSARLDIQPIGVGGFRSAHSQAFAKNVGEN | |
| sequence | FVNLMVYALACILKDNDDVLVDKGLPPHLKKALTLSRE | |
| | CRIKDTLREIKIPIEGDLCVFSRSNHCNAIVISAKTRL | |
| | KEVFHIGTMWALFSDVAKDEYCLNKWGLKVESSESLKD | |
| | TMYVFATADMINKDGARSQGCDVERETPRNLIAMDASF | |
| | FDYVFVSKMGIGHVSSDLSLKYGRESLFHELGCIIDMI | |
| | EQKFDILL | |
| pET21- | TGGCGAATGGGACGCGCCCTGTAGCGGCGCATT | 3 |
| R.PfoI | AAGCGCGGCGGGTGTGGTGGTTACGCGCAGCG | |
| construct | TGACCGCTACACTTGCCAGCGCCCTAGCGCCCGC | |
| | TCCTTTCGCTTTGTTCCCTTCCTTTCTCGCCACGTT | |
| | CGCCGGCTTTCCCCGTCAAGCTCTAAATCGGGGG | |
| | CTCCCTTTAGGGTTCCGATTTAGTGCTTTACGGC | |
| | ACCTCGACCCCAAAAAACTTGATTAGGGTGATGG | |
| | TTCACGTAGTGGGCCATCGCCCTGATAGACGGTT | |
| | TTTCGCCCTTTGACGTTGGAGTCCACGTTCTTTA | |
| | ATAGTGGACTCTTGTTCCAAACTGGAACAACACT | |
| | CAACCCTATCTCGGTCTATTCTTTTGATTTATAAG | |
| | GGATTTTGCCGATTTCGGCCTATTGGTTAAAAAA | |
| | TGAGCTGATTTAACAAAAATTTAACGCGAATTTT | |
| | AACAAAATATTAACGTTTACAATTTCAGGTGGCA | |
| | CTTTTCGGGGAAATGTGCGCGGAACCCCTATTTG | |
| | TTTATTTTTCTAAATACATTCAAATATGTATCCGC | |
| | TCATGAGACAATAACCCTGATAAATGCTTCAATA | |
| | ATATTGAAAAAGGAAGAGTATGAGTATTCAACAT | |
| | TTCCGTGTCGCCCTTATTCCCTTTTTTGCGGCATT | |
| | TTGCCTTCCTGTTTTTGCTCACCCAGAAACGCTG | |
| | GTGAAAGTAAAAGATGCTGAAGATCAGTTGGGT | |
| | GCACGAGTGGGTTACATCGAACTGGATCTCAACA | |
| | GCGGTAAGATCCTTGAGAGTTTTCGCCCCGAAG | |
| | AACGTTTTCCAATGATGAGCACTTTTAAAGTTCT | |
| | GCTATGTGGCGCGGTATTATCCCGTATTGACGCC | |
| | GGGCAAGAGCAACTCGGTCGCCGCATACACTATT | |
| | CTCAGAATGACTTGGTTGAGTACTCACCAGTCAC | |
| | AGAAAAGCATCTTACGGATGGCATGACAGTAAG | |
| | AGAATTATGCAGTGCTGCCATAACCATGAGTGAT | |
| | AACACTGCGGCCAACTTACTTCTGACAACGATCG | |
| | GAGGACCGAAGGAGCTAACCGCTTTTTTGCACAA | |
| | CATGGGGGATCATGTAACTCGCCTTGATCGTTG | |
| | GGAACCGGAGCTGAATGAAGCCATACCAAACGA | |
| | CGAGCGTGACACCACGATGCCTGCAGCAATGGC | |
| | AACAACGTTGCGCAAACTATTAACTGGCGAACTA | |
| | CTTACTCTAGCTTCCCGGCAACAATTAATAGACT | |
| | GGATGGAGGCGGATAAAGTTGCAGGACCACTTC | |
| | TGCGCTCGGCCCTTCCGGCTGGCTGGTTTATTGC | |
| | TGATAAATCTGGAGCCGGTGAGCGTGGGTCTCG | |
| | CGGTATCATTGCAGCACTGGGGCCAGATGGTAA | |
| | GCCCTCCCGTATCGTAGTTATCTACACGACGGGG | |
| | AGTCAGGCAACTATGGATGAACGAAATAGACAG | |
| | ATCGCTGAGATAGGTGCCTCACTGATTAAGCATT | |
| | GGTAACTGTCAGACCAAGTTTACTCATATATACT | |
| | TTAGATTGATTTAAAACTTCATTTTTAATTTAAAA | |
| | GGATCTAGGTGAAGATCCTTTTTGATAATCTCAT | |
| | GACCAAAATCCCTTAACGTGAGTTTTCGTTCCACT | |
| | GAGCGTCAGACCCCGTAGAAAAGATCAAAGGAT | |
| | CTTCTTGAGATCCTTTTTTTCTGCGCGTAATCTGC | |
| | TGCTTGCAAACAAAAAAACCACCGCTACCAGCGG | |
| | TGGTTTGTTTGCCGGATCAAGAGCTACCAACTCT | |
| | TTTTCCGAAGGTAACTGGCTTCAGCAGAGCGCA | |
| | GATACCAAATACTGTCCTTCTAGTGTAGCCGTAG | |
| | TTAGGCCACCACTTCAAGAACTCTGTAGCACCGC | |
| | CTACATACCTCGCTCTGCTAATCCTGTTACCAGTG | |
| | GCTGCTGCCAGTGGCGATAAGTCGTGTCTTACC | |
| | GGGTTGGACTCAAGACGATAGTTACCGGATAAG | |
| | GCGCAGCGGTCGGGCTGAACGGGGGGTTCGTG | |
| | CACACAGCCCAGGTTGGAGCGAACGACCTACACC | |
| | GAACTGAGATACCTACAGCGTGAGCTATGAGAA | |
| | AGCGCCACGCTTCCCGAAGGGAGAAAGGCGGAC | |
| | AGGTATCCGGTAAGCGGCAGGGTCGGAACAGG | |
| | AGAGCGCACGAGGGAGCTTCCAGGGGGAAACG | |
| | CCTGGTATCTTTATAGTCCTGTCGGGTTTCGCCA | |
| | CCTCTGAGTTGAGCGTCGATTTTTGTGATGCTCG | |
| | TCAGGGGGGCGGAGCCTATGGAAAAACGCCAGC | |
| | AACGCGGCCTTTTTACGGTTCCTGGCCTTTTGCT | |
| | GGCCTTTTGCTCACATGTTCTTTCCTGCGTTATCC | |
| | CCTGATTCTGTGGATAACCGTATTACCGCCTTTG | |
| | AGTGAGCTGATACCGCTCGCCGCAGCCGAACGA | |
| | CCGAGCGCAGCGAGTCAGTGAGCGAGGAAGCG | |
| | GAAGAGCGCCTGATGCGGTATTTTCTCCTTACGC | |
| | ATCTGTGCGGTATTTCACACCGCATATATGGTGC | |
| | ACTCTCAGTACAATCTGCTCTGATGCCGCATAGT | |
| | TAAGCCAGTATACACTCCGCTATCGCTACGTGAC | |
| | TGGGTCATGGCTGCGCCCCGACACCCGCCAACAC | |
| | CCGCTGACGCGCCCTGACGGGCTTGTCTGCTCCC | |
| | GGCATCCGCTTACAGACAAGCTGTGACCGTCTCC | |
| | GGGAGCTGCATGTGTCAGAGGTTTTCACCGTCA | |
| | TCACCGAAACGCGCGAGGCAGCTGCGGTAAAGC | |
| | TCATCAGCGTGGTCGTGAAGCGATTCACAGATG | |
| | TCTGCCTGTTCATCCGCGTCCAGCTCGTTGAGTT | |
| | TCTCCAGAAGCGTTAATGTCTGGCTTCTGATAAA | |
| | GCGGGCCATGTTAAGGGCGGTTTTTTCCTGTTTG | |
| | GTCACTGATGCCTCCGTGTAAGGGGGATTTCTGT | |
| | TCATGGGGGTAATGATACCGATGAAACGAGAGA | |
| | GGATGCTCACGATACGGGTTACTGATGATGAAC | |
| | ATGCCCGGTTACTGGAACGTTGTGAGGGTAAAC | |
| | AACTGGCGGTATGGATGCGGCGGGACCAGAGA | |
| | AAAATCACTCAGGGTCAATGCCAGCGCTTCGTTA | |
| | ATACAGATGTAGGTGTTCCACAGGGTAGCCAGC | |
| | AGCATCCTGCGATGCAGATCCGGAACATAATGGT | |
| | GCAGGGCGCTGACTTCCGCGTTTCCAGACTTTAC | |
| | GAAACACGGAAACCGAAGACCATTCATGTTGTTG | |
| | CTCAGGTCGCAGACGTTTTGCAGCAGCAGTCGCT | |
| | TCACGTTCGCTCGCGTATCGGTGATTCATTCTGC | |
| | TAACCAGTAAGGCAACCCCGCCAGCCTAGCCGG | |
| | GTCCTCAACGACAGGAGCACGATCATGCGCACCC | |
| | GTGGGGCCGCCATGCCGGCGATAATGGCCTGCT | |
| | TCTCGCCGAAACGTTTGGTGGCGGGACCAGTGA | |
| | CGAAGGGTTGAGCGAGGGCGTGCAAGATTCCGA | |
| | ATACCGCAAGCGACAGGCCGATCATCGTCGCGCT | |
| | CCAGCGAAAGCGGTCCTCGCCGAAAATGACCCA | |
| | GAGCGCTGCCGGCACCTGTCCTACGAGTTGCAT | |
| | GATAAAGAAGACAGTCATAAGTGCGGCGACGAT | |
| | AGTCATGCCCCGCGCCCACCGGAAGGAGCTGAC | |
| | TGGGTTGAAGGCTCTCAAGGGCATCGGTCGAGA | |
| | TCCCGGTGCCTAATGAGTGAGCTAACTTACATTA | |
| | ATTGCGTTGCGCTCACTGCCCGCTTTCCAGTCGG | |
| | GAAACCTGTCGTGCCAGCTGCATTAATGAATCGG | |
| | CCAACGCGCGGGGAGAGGCGGYTTGCGTATTGG | |
| | GCGCCAGGGTGGTTTTTCTTTTCACCAGTGAGAC | |
| | GGGCAACAGCTGATTGCCCTTCACCGCCTGGCCC | |
| | TGAGAGAGTTGCAGCAAGCGGTCCACGCTGGTT | |
| | TGCCCCAGCAGGCGAAAATCCTGTTTGATGGTG | |
| | GTTAACGGCGGGATATAACATGAGCTGTGTTCG | |
| | GTATCGTCGTATCCCACTACCGAGATATCCGCAC | |
| | CAACGCGCAGCCCGGACTCGGTAATGGCGCGCA | |
| | TTGCGCCCAGCGCCATCTGATCGTTGGCAACCAG | |
| | CATCGCAGTGGGAACGATGCCCTCATTCAGCATT | |
| | TGCATGGTTTGTTGAAAACCGGACATGGCACTCC | |
| | AGTCGCCTTCCCGTTCCGCTATCGGCTGAATTTG | |
| | ATTGCGAGTGAGATATTTATGCCAGCCAGCCAGA | |
| | CGCAGACGCGCCGAGACAGAACTTAATGGGCCC | |
| | GCTAACAGCGCGATTTGCTGGTGACCCAATGCG | |
| | ACCAGATGCTCCACGCCCAGTCGCGTACCGTCTT | |
| | CATGGGAGAAAATAATACTGTTGATGGGTGTCT | |
| | GGTCAGAGACATCAAGAAATAACGCCGGAACAT | |
| | TAGTGCAGGCAGCTTCCACAGCAATGGCATCCTG | |
| | GTCATCCAGCGGATAGTTAATGATCAGCCCACTG | |
| | ACGCGTTGCGCGAGAAGATTGTGCACCGCCGCT | |
| | TTACAGGCTTCGACGCCGCTTCGTTCTACCATCG | |
| | ACACCACCACGCTGGCACCCAGTTGATCGGCGCG | |
| | AGATTTAATCGCCGCGACAATTTGCGACGGCGC | |
| | GTGCAGGGCCAGACTGGAGGTGGCAACGCCAAT | |
| | CAGCAACGACTGTTTGCCCGCCAGTTGTTGTGCC | |
| | ACGCGGTTGGGAATGTAATTCAGCTCCGCCATCG | |
| | CCGCTTCCACTTTTTCCCGCGTTTTCGCAGAAACG | |
| | TGGCTGGCCTGGTTCACCACGCGGGAAACGGTC | |
| | TGATAAGAGACACCGGCATACTCTGCGACATCGT | |
| | ATAACGTTACTGGTTTCACATTCACCACCCTGAAT | |
| | TGACTCTCTTCCGGGCGCTATCATGCCATACCGC | |
| | GAAAGGTTTTGCGCCATTCGATGGTGTCCGGGA | |
| | TCTCGACGCTCTCCCTTATGCGACTCCTGCATTAG | |
| | GAAGCAGCCCAGTAGTAGGTTGAGGCCGTTGAG | |
| | CACCGCCGCCGCAAGGAATGGTGCATGCAAGGA | |
| | GATGGCGCCCAACAGTCCCCCGGCCACGGGGCC | |
| | TGCCACCATACCCACGCCGAAACAAGCGCTCATG | |
| | AGCCCGAAGTGGCGAGCCCGATCTTCCCCATCG | |
| | GTGATGTCGGCGATATAGGCGCCAGCAACCGCA | |
| | CCTGTGGCGCCGGTGATGCCGGCCACGATGCGT | |
| | CCGGCGTAGAGGATCGAGATCTCGATCCCGCGA | |
| | AATTAATACGACTCACTATAGGGGAATTGTGAGC | |
| | GGATAACAATTCCCCTCTAGAAATAATTTTGTTTA | |
| | ACTTTAAGAAGGAGATATACATATGCAGAAGTAT | |
| | AGGTTGTATGAGAAGGATGGCTCGCCTGTTCAA | |
| | GACTTCAATCGATTTGTAAAAGGCTGGTTAGATA | |
| | TTGAGTTTGGTTTAAAGGAGCATCAGCCGCCAAA | |
| | GGTATTTGATACTATAAGAGATAAGTATAACGAA | |
| | GCTATAGAAGCTGTTGTATTGAGCGGTGTTGCG | |
| | CCGCGGACGGCACATAAGGCTGCGCTTTCCACTT | |
| | TGACCGAACTTCTATTTGGTCATGATCTTGCAAA | |
| | AGAACTTAGTGCTCGTTTGGATATTCAGCCCATA | |
| | GGAGTAGGCGGATTTCGTTCGGCGCATTCTCAA | |
| | GCATTTGCTAAAAATGTTGGCGAGAACTTTGTAA | |
| | ATTTAATGGTCTATGCGCTTGCGTGTATCTTGAA | |
| | AGATAATGATGATGTTCTGGTAGATAAGGGGCT | |
| | GCCACCTCATCTAAAAAAAGCTCTAACATTGAGT | |
| | CGTGAGTGTAGGATTAAAGATACGCTTCGTGAA | |
| | ATAAAGATCCCTATAGAAGGGGATCTGTGTGTTT | |
| | TTTCACGAAGTAATCATTGCAATGCAATCGTTAT | |
| | TAGCGCTAAAACAAGACTCAAAGAAGTCTTTCAT | |
| | ATTGGCACAATGTGGGCGTTGTTTTCTGATGTG | |
| | GCGAAAGATGAATACTGTCTTAACAAGTGGGGG | |
| | CTGAAAGTTGAGTCGTCAGAGTCCCTCAAGGATA | |
| | CGATGTATGTATTTGCTACAGCTGATATGATAAA | |
| | TAAAGATGGTGCAAGAAGTCAGGGTTGTGATGT | |
| | GGAGAGGGAAACTCCAAGAAACTTAATTGCGAT | |
| | GGACGCCTCTTTTTTTGATTATGTTTTTGTTTCGA | |
| | AGATGGGTATCGGGCATGTCTGTTCCGATCTTTC | |
| | TCTTAAGTATGGGCGAGAATCGCTGTTCCACGAG | |
| | CTAGGGTGCATAATTGATATGATCGAACAAAAGT | |
| | TTGATATTTTACTTTAAAATCACTTTTCCTCCGTC | |
| | CTCCGTCGACAAGGTTGCGGCCGCACTCGAGCAC | |
| | CACCACCACCACCACTGAGATCCGGCTGCTAACA | |
| | AAGCCCGAAAGGAAGCTGAGTTGGCTGCTGCCA | |
| | CCGCTGAGCAATAACTAGCATAACCCCTTGGGGC | |
| | CTCTAAACGGGTCTTGAGGGGTTTTTTGCTGAAA | |
| | GGAGGAACTATATCCGGAT | |
| Primer | ATCCCAGCACCATTCACAACCC | 4 |
| for PCR | | |
| Primer | GCATCCACTACGCCCGCTGCAC | 5 |
| for PCR | | |
| Primer | GAGGGCATATGCAGAAGTATAGGTTGTATG | 6 |
| for | | |
| amplifi- | | |
| cation | | |
| Primer | GAAAAGTGATTTTAAAGTAAAATATC | 7 |
| for | | |
| amplifi- | | |
| cation | | |
| M.Ecl18kI | ATGACTGA TAATATCGCA GCAACAATCA | 8 |
| nucleic | AAGAAAAAAG AGAAAGACTT CATATGACTC | |
| acid | AAAAAGAGTT TGCAGACGCC CTTGGTCTTT | |
| sequence | CAAAATATGG AGACAGAACC ATAAGAAGAT | |
| | GGGAGCGCGG GGAAACAAAA CCTACTGGGG | |
| | CGGAACTAAA AGCAGTTATG GACTTTCCAG | |
| | ACACACCACC TTACCCAAAC AACGAGAATG | |
| | GCAGATATCG CATGATCGAT TTATTTGCTG | |
| | GAATCGGTGG AACCAGACTT GGATTTCATC | |
| | AAACTAACGC TGTAAATGTT GTTTTTTCCA | |
| | GCGAATGGGA TAAGTTTGCT CAAAAGACAT | |
| | ATCATGCAAA TTATGGTGAT TTCCCTGATG | |
| | GGGACATAAC AAAAATCGAT GAAAAAGATA | |
| | TTCCTGATCA TGAAATTCTT GTCGGTGGGT | |
| | TTCCTTGCGT AGCATTCTCT CAAGCAGGGC | |
| | TCAAAAAAGG ATTTAATGAT ACAAGAGGAA | |
| | CTTTGTTTTT TGATATAGCA AGGATAATAA | |
| | AGGAAAAAAA ACCTCATGCT TTTTTACTTG | |
| | AAAATGTAAA AAACTTGCTA GGGCATGATA | |
| | AAGGTAGAAC TTTTTCAATA ATAAAAAATA | |
| | CACTTGAAGA ATTGAATTAC ACTGTGTATT | |
| | ACAATATCTT CGCTGCAAAA GACTTTGGTG | |
| | TTCCACAAAA TAGAGAGCGC ATATACATTG | |
| | TAGGCTTTAA TAAAGAAAAA GTAAGAAATC | |
| | ATGAACATTT TACTTTTCCA ACCCCATIAA | |
| | AAACAAAAAC AAGAGTTGGA GATATTCTTG | |
| | AAAAGAGCGT AGACAATAAA TATACGCTAT | |
| | CAGATGCGCT CTGGAATGGA CACCAACGCA | |
| | GGAAACTTGT AAATGCAGCA GCCGGTAAAG | |
| | GCTTTGGGTA TGGTTTATTT AATGAAAATA | |
| | GCCCATATAC TAATACCATA AGTGCGAGGT | |
| | ACTATAAAGA TGGTAGCGAA ATATTAATAG | |
| | AGCAAAAAGG AAGTAACCCA AGAAAAATAA | |
| | CTCCGCGGGA AGCGTCAAGA CTTCAAGGGT | |
| | TTCCTAGTGA TTTTATTATC CCTGTATCGG | |
| | ATACACAGGC TTATAAGCAA TTTGGGAATT | |
| | CAGTCGCAGT CCCTGTTATC AATGCAATAG | |
| | CAGAAAAAAT CATATCGACC TTAGATTCA | |
| Amino | MAVRRKARLYEDGVPCENFNQRIKSWLDMTETIK | 9 |
| acid | EHTTPKAFESLRQLYTQNLDELLLISEDPNDGWSA | |
| sequence | HRHLLSNLTDVMYGIDIAVELATRLSPDSINSSGF | |
| of | RTALSQGAAKNTGENFINIIVYILADLLSYQDEVL | |
| hypothe- | VVKGLPPMLREPLTLRRTFTDKNGVMREIKMPIEG | |
| tical | DLCIFSRSNPLNAIVVNAKTRLKFIFHIGTMWKIF | |
| protein | FDMVDDDYLLNKWGLRKDPNLTTALSTNMQYIFTT | |
| [Bacillus | ADMIKTDGTNTQGGDVEREDVRNLIAMDASFFDYV | |
| sp. | FVSKQNISHVANSIEISSGREALFHELGCLLDMIK | |
| Leaf13] | QKYNI | |

### DESCRIPTION OF THE EMBODIMENTS

This application describes the successful expression of recombinant R.PfoI in E. coli. Present embodiments relate to recombinant DNA of SEQ ID NO: 2 that encodes R.PfoI and recombinant DNA of SEQ ID NO: 8 that encodes M.Ec118kI in host cells, and to the expression of the PfoI in E. coli cells that contain the recombinant DNA.

### I. Definitions

“Modifying,” as used herein, refers to inducing a change in a nucleic acid structure. For example, methylation enzymes can methylate DNA and thus modify the nucleic acid structure.

“Premodifying,” as used herein, refers to modifying that occurs before expression of a restriction endonuclease. For example, methylation of restriction endonuclease cleavage sites in the genome of a host cell before expression of the restriction endonuclease may allow expression of a recombinant restriction endonuclease in a host cell without damage to the host cell DNA. In the absence of premodifying of the host cell genome, expression of a restriction endonuclease can be toxic to the host cell by causing cleavage of the host genome. Premodification can protect all restriction endonuclease sites and hence prevent all cleavage of host genome DNA or it can protect a portion of the restriction endonuclease sites and hence provide useful protection (even if not complete) against cleavage of host genome DNA.

“pfoIM,” as used herein, refers to the gene that expresses M.PfoI.

“pfoIR,” as used herein, refers to the gene that expresses R.PfoI. pfoIR comprises the sequence in SEQ ID NO: 1.

“M.PfoI,” as used herein, refers to the methylation enzyme that is normally protective against the restriction endonuclease PfoI in Pseudomonas fluorescens biovar 126 to protect genomic DNA of Pseudomonas fluorescens biovar 126. M.PfoI may be referred to as the cognate methylase for PfoI.

“R.PfoI,” as used herein, refers to the restriction endonuclease PfoI. The amino acid sequence of R.PfoI comprises SEQ ID NO: 2. The characteristics of natively-expressed PfoI from Pseudomonas fluorescens biovar 126 has been described in Gaigalas et al., Nucleic Acids Research, 30(19):e98, 2002; although the nucleic acid sequence encoding PfoI nor the amino acid sequence of PfoI was not known. For this reason, the prior R.PfoI was limited to the native endonuclease isolated from Pseudomonas fluorescens biovar 126, which lead to less convenient expression. Pseudomonas fluorescens biovar 126 strain was identified by Fermentas UAB and is currently controlled by Thermo Fisher Scientific Baltics UAB. The strain is not publicly available and has not been deposited in any public databases. The art has had a need for the nucleic acid sequence of R.PfoI so that it could be expressed recombinantly.

“M.Ec118kI,” as used herein, is the methylation enzyme associated with the Ec118kI restriction endonuclease. The sequence of the gene encoding M.Ec118kI is SEQ ID NO: 8.

“Host cell,” as used herein, refers to a cell into which exogenous DNA (recombinant) has been introduced. In some embodiments, host cells include any prokaryotic and eukaryotic cells suitable for expressing an exogenous DNA (e.g., a recombinant nucleic acid sequence). Exemplary host cells include E. coli, bacterial expression cells, yeast expression cells, algae expression cells, insect expression cells, mammalian expression cells, and cell-free in vitro expression systems (allowing for rapid expression directly from a DNA template (e.g. a linear or circular plasmid, a PCR product or other DNA fragment that contains a promoter sequence upstream of the gene to be transcribed) or mRNA template). One skilled in the art would know multiple hosts suitable for protein (restriction endonuclease) expression. A host cell does not include the organism that naturally contains the nucleic acid sequence of interest or that naturally produces the protein of interest.

“Restriction enzyme,” “endonuclease,” or “restriction endonuclease,” as used herein, refers to an endonuclease that cuts DNA at or near a specific recognition nucleotide sequence known as a restriction site.

“Methylation” or “restriction endonuclease methylation,” as used here refers to when methylation of a nucleic acid blocks recognition or cutting by a restriction endonuclease. A methylation enzyme that blocks recognition and/or cleavage by a particular restriction endonuclease may be termed a cognate methylase. A cognate methylase may be expressed by a cell that normally expresses a restriction endonuclease to protect genomic DNA from cleavage by this restriction endonuclease. A non-cognate methylase is one that is not expressed natively in the same cell as a restriction endonuclease. However, a non-cognate methylase may be able to serve a protective role in a host cell and allow expression of a recombinant restriction endonuclease by methylating DNA to block recognition of genomic DNA of the host cell by the recombinant restriction endonuclease.

“Palindromic sequence,” as used herein refers to when a nucleic acid sequence on double-stranded DNA or RNA wherein reading 5′-3′ on one strand matches the sequence reading backwards 5′-3′ on the complementary strand when it forms a double helix.

“Recombinant,” as used herein, refers to an exogenously expressed protein.

“Vector,” as used herein, refers to a DNA molecule used as a vehicle to artificially carry foreign genetic material into another cell, where it can be replicated and/or expressed. An example vector would be a plasmid, although one skilled in the art would be familiar with a wide variety of vectors.

### II. Recombinant PfoI Restriction Endonuclease

This application describes recombinant PfoI endonuclease. Vectors and host cells for expression of recombinant PfoI are also described.

A. PfoI Endonuclease

PfoI is restriction endonuclease that is produced by the bacterium Pseudomonas fluorescens biovar 126. The amino acid sequence of PfoI is SEQ ID NO: 2. PfoI recognizes an interrupted hexanucleotide palindromic sequence 5′-T↓CCNGGA-3′ and cleaves DNA to produce protruding pentanucleotide 5′-ends (Gaigalas et al., Nucleic Acids Research, 30(19):e98 (2002)).

Although the native PfoI restriction endonuclease is known, it is provided only by one commercial supplier—Thermo Fisher Scientific, as the strain is proprietary and not publicly available. There is a continuing need for recombinant endonuclease which offers significantly larger yields, greater product consistency, and less lot-to-lot variation than those produced by native strains. Once an endonuclease system is cloned, choice of expression vector and host cell allows tight control over the production environment enabling increased product quality and purity. However, it is not necessarily straightforward to clone an endonuclease and determine its nucleic acid sequence even when the endonuclease is known.

In some embodiments, the nucleic acid sequence of R.PfoI is SEQ ID NO: 1.

One skilled in the art would be aware that conservative mutations in the sequence of recombinant R.PfoI (SEQ ID NO: 1) are unlikely to affect the function of the PfoI restriction endonuclease.

B. Recombinant Vectors

In some embodiments, a recombinant vector allows replication and/or expression of recombinant PfoI. In some embodiments, a recombinant vector may be referred to as an expression vector.

In some embodiments, a recombinant vector can carry the nucleic acid sequence of PfoI (SEQ ID NO: 1) into a cell where the PfoI restriction endonuclease is not normally expressed. In some embodiments, a recombinant vector can carry the nucleic acid sequence of PfoI (SEQ ID NO: 1) into a cell where this sequence in not normally comprised in the cell's genome. In some embodiments, a recombinant vector can carry the nucleic acid sequence of PfoI (SEQ ID NO: 1) into a cell that is not Pseudomonas fluorescens biovar 126.

In some embodiments, a recombinant vector comprises the nucleic acid sequence of PfoI (SEQ ID NO: 1).

In some embodiments, a recombinant vector comprises a nucleic acid that encodes SEQ ID NO: 2.

In some embodiments, a recombinant vector comprises a nucleic acid that encodes a restriction endonuclease polypeptide with at least 90% sequence identity to SEQ ID NO: 2. In some embodiments, a recombinant vector comprises a nucleic acid that encodes a polypeptide with at least 95% sequence identity to SEQ ID NO: 2. In some embodiments, a recombinant vector comprises a nucleic acid that encodes a polypeptide with at least 97% sequence identity to SEQ ID NO: 2. In some embodiments, a recombinant vector comprises a nucleic acid that encodes a polypeptide with at least 99% sequence identity to SEQ ID NO: 2.

In some embodiments, a recombinant vector further comprises at least one regulatory element that is not from Pseudomonas fluorescens. Thus, in some embodiments, the recombinant vector comprises DNA encoding PfoI from Pseudomonas fluorescens and DNA encoding at least one regulatory element from a different organism, making a nonnaturally-occurring sequence.

In some embodiments, a recombinant vector comprises at least one regulatory element that is not present in the bacterium Pseudomonas fluorescens biovar 126.

In some embodiments, the regulatory element comprises at least one of a promoter, a translation initiation sequence, a termination codon, a transcription termination sequence, or a sequence encoding a protein tag.

In some embodiments, the recombinant vector is a plasmid. One skilled in the art would be familiar with a wide variety of vectors, and this invention is not limited to use of a specific vector or to any specific regulatory elements.

In some embodiments, the recombinant vector comprising said nucleic acid of interest is codon-optimized for expression by a host organism. Methods for optimizing codons based on the degeneracy of genetic code improves expression of heterologous genes in various hosts, and are well known in the art. A variety of codon optimization tools are available that help determine codon usage and codon bias. Exemplary codon optimization tools are available online, for e.g., from GeneArt, Genscript or IDT, etc. Alternatively, codon optimization is provided as part of a service for gene synthesis, for e.g., by GeneArt (Thermo Fisher Scientific), etc. Therefore, one skilled in the art would be familiar with a variety of methods and tools for codon optimization of nucleic acid sequences. In some embodiments, a nucleic acid that encodes for SEQ ID NO: 2, or, a nucleic acid that encodes for a restriction endonuclease polypeptide with at least 90%, 95%, 97%, or 99% sequence identity to SEQ ID NO: 2, are codon-optimized for expression by a host organism.

C. Host Cells

In some embodiments, a recombinant vector comprising the nucleic acid sequence of PfoI (SEQ ID NO: 1) is introduced into a host cell. In some embodiments, the host cell is suitable for expressing the amino acid sequence of PfoI (SEQ ID NO: 2) encoded by a recombinant vector. The host cell can be any cell besides Pseudomonas fluorescens biovar 126.

In some embodiments, host cells allow for expression of recombinant PfoI (SEQ ID NO: 2). In some embodiments, host cells express protein encoded by a recombinant vector comprising the nucleic acid sequence of PfoI (SEQ ID NO: 1).

In some embodiments, a host cell allows for production and purification of recombinant PfoI.

In some embodiments, a host is transformed by a recombinant vector according to Section II.B.

In some embodiments, a host cell is transformed with a recombinant vector comprising a nucleic acid of SEQ ID NO: 1, and a restriction endonuclease encoded by SEQ ID NO: 1 binds to the nucleotide sequence TCCNGGA and cleaves between T and C in each strand, producing DNA fragments with protruding pentanucleic 5′-ends.

In some embodiments, the host cell is a prokaryotic or eukaryotic cell.

In some embodiments, the host cell is E. coli, bacterial expression cells, yeast expression cells, algae expression cells, insect expression cells, or mammalian expression cells. In some embodiments, the host cell is E. coli.

Included in host cells is also cell-free in vitro expression systems (allowing for rapid expression directly from a DNA template (e.g. a linear or circular plasmid, a PCR product or other DNA fragment that contains a promoter sequence upstream of the gene to be transcribed) or mRNA template). In some embodiments, a cell-free in vitro expression system is used in place of a host cell. In any method comprising a host cell, a cell-free in vitro expression system may be substituted for the host cell.

One skilled in the art would know multiple host cells suitable for restriction endonuclease expression, and this invention is not limited to use of a specific host cell.

### III. Methods of Production of Recombinant PfoI

This application also describes methods of producing recombinant PfoI restriction endonuclease from a host cell.

In some embodiments, the methods comprise culturing the host cell according to section II.C under conditions for expression of PfoI restriction endonuclease.

In some embodiments, a host cell is transformed with a recombinant vector comprising a nucleic acid of SEQ ID NO: 1, and a restriction endonuclease encoded by SEQ ID NO: 1 binds to the nucleotide sequence TCCNGGA and cleaves between T and C in each strand, producing DNA fragments with protruding pentanucleic 5′-ends.

Conditions for expressing a recombinant protein, such as PfoI restriction endonuclease, in a host cell would be well-known to those skilled in the art. For example, one skilled in the art would be well-aware of method of constitutive or induced recombinant protein production from E. coli, as well as other host cells.

A. Premodifying Host Cell DNA

Although expression of recombinant proteins in a host cell are well-known, expression of recombinant restriction endonucleases poses unique difficulties even to those skilled in the art. When expressed in host cells, restriction endonucleases can cut the host genome at restriction sites and lead to host cell toxicity. This occurs because the host cell is not the native cell for the restriction endonuclease and does not have natural protection mechanisms for expression of restriction endonucleases. Thus, expression of a restriction endonuclease may require specific steps for premodifying host cell DNA.

In some embodiments, host cells are premodified to improve expression of PfoI. In some embodiments, host cells are premodified to reduce cutting of the host cell genome by PfoI. In some embodiments, host cells are premodified to reduce toxicity induced by PfoI expression in host cells. In some embodiments, PfoI will not be expressed at sufficient amounts in the absence of premodification of host cells. As discussed above, premodification may be complete or it may be partial, but at a sufficient level to allow for a functioning host cell expression system.

In some embodiments, host cell DNA is modified at one or more nucleotides in the PfoI restriction site to protect from cutting of the DNA by PfoI. In some embodiments, host cell DNA of sequence TCCNGGA is modified at one or more nucleotides. In some embodiments, host cell DNA of sequence TCCNGGA is methylated at one or more nucleotides.

In some embodiments, premodifying of the host cell DNA is achieved by expressing an enzyme that can modify DNA at one or more nucleotides of the PfoI restriction site. In some embodiments, premodifying of the host cell DNA is achieved by expressing an enzyme that can methylate at one or more nucleotides of the sequence TCCNGGA. In some embodiments, the premodifying of the host cell DNA is achieved by expressing the M.Ec118kI methylation enzyme. In some embodiments, the premodifying of the host cell DNA is achieved by transformation with a recombinant vector comprising SEQ ID NO: 8. In some embodiments, the premodifying of the host cell DNA is achieved by transformation with a recombinant vector comprising SEQ ID NO: 8 and causing methylation of host cell DNA by culturing the host cell under conditions for expression of M.Ec118kI.

In some embodiments premodified host cells are cultured under conditions for expression of PfoI restriction endonuclease.

### EXAMPLES

### Example 1. Preparation of Genomic DNA

In order to clone the genes encoding the PfoI restriction-modification system, genomic DNA of Pseudomonas fluorescens biovar 126 was prepared. The isolated genomic DNA of Pseudomonas fluorescens biovar 126 was sonicated. The resulting DNA fragments were fractionated on agarose gel. DNA fragments 3-10 kb in size were isolated, ends of molecules were blunted with the T4 DNA polymerase in the presence of deoxyribonucleotides.

### Example 2. Construction of a Plasmid for Selection

Resulting fragments following preparation of genomic DNA were ligated to Eco47III-cleaved and dephosphorylated positive selection vector pJRD-R (Ausubel et al., Current Protocols in Molecular Biology (1987); Sambrook et al., Molecular Cloning: A Laboratory Manual. Cold Spring Harbor, N.Y., Cold Spring Harbor Laboratory Press (1989); Cesnaviciene et al., J Mol Biol 314: 205-16 (2001)). Prior to ligation vector pJRD-R was modified by inserting two DNA fragments bearing PfoI recognition sequences to enable biochemical selection of PfoI R-M enzymes. The ligation mixture was used to electrotransform E. coli ER2267 cells. Transformed cells were plated on LB medium with ampicillin and chloramphenicol and grown overnight.

### Example 3. Restriction Digestion of Cloned Genomic DNA Library

Plasmid DNA was isolated from the pooled 130,000 ampicillin and chloramphenicol resistant colonies. The biochemical selection (Szomolanyi et al., Gene 10: 219-25 (1980)) was used to select the clones possessing PfoI methyltransferase activity. To perform the selection, plasmid DNA was digested with an excess of PfoI restriction endonuclease and used to transform E. coli ER2267. Then, randomly picked transformant colonies were screened for resistance to PfoI restriction endonuclease digestion. Plasmid DNAs of 6 clones out of the 87 tested were found to be partially resistant to PfoI digestion. Plasmid DNA from one clone, that was found to be partially resistant to PfoI digestion (pJRD-PfoM6), was selected for further analysis.

The cloned 3.9 kb DNA fragment was sequenced. DNA sequence analysis demonstrated the presence of two open reading frames (ORFs). ORF no. 1 (2103 bp in length) was proposed to encode PfoI methyltransferase.

Amino acid sequence analysis of the protein, encoded by the ORF no. 2 (1248 bp in length) demonstrated similarity with ribonucleotide-diphosphate reductase beta subunit.

### Example 4. Inverse PCR Amplification of pfoIR Gene

Inverse PCR (Sambrook et al., 1989) was used to clone the genomic DNA fragment encoding PfoI restriction endonuclease gene. To perform inverse PCR Pseudomonas fluorescens biovar 126 genomic DNA was digested with HindIII restriction endonuclease. Restriction fragments were self-ligated. Ligation product was used as a template for PCR with the following primers:

(SEQ ID NO. 4)
5′-atcccagcaccattcacaaccc
(SEQ ID NO. 5)
5′-gcatccactacgcccgctgcac

PCR product was fractionated on the agarose gel. 2.3 kb PCR fragment was digested with Call restriction endonuclease, the resulted fragment ends were blunted and phosphorylated. 1 kb DNA fragment was isolated from agarose gel and ligated into SmaI-cleaved and dephosphorylated pUC19 vector (Yanisch-Perron, C. et al, Gene 33: 103-19 (1985)). The ligation mixture was used to transform E. coli ER2267 cells. The nucleotide sequence of cloned 1 kb DNA fragment was determined by sequencing of plasmid DNA of 4 ampicillin-resistant clones. DNA sequence analysis demonstrated the presence of open reading frame (ORF) no. 3 (936 bp in length). ORF no. 3 (SEQ ID NO: 1) was proposed to encode PfoI restriction endonuclease.

### Example 5. Strategy to Co-Express pfoIM Gene and pfoIR in E. coli

To express pfoIR gene, DNA fragment encompassing the ORF no. 3 was amplified from Pseudomonas fluorescens biovar 126 genomic DNA using the following primers:

(SEQ ID NO: 6)
5′-gagggcatatgcagaagtataggttgtatg
(SEQ ID NO: 7)
5′-gaaaagtgattttaaagtaaaatatc

The resulting 0.95 kb PCR fragment was digested with NdeI restriction endonuclease, phosphorylated and ligated into NdeI-Ec1136II digested and dephosphorylated pET21b(+) vector under control of the T7lac promoter generating pET-R.PfoI. DH10B carrying PfoI methyltransferase gene in plasmid pJRD-PfoM6 [Kn^{r}, Cm^{r}, Ap^{r}] was used as a host for transformation of pET21b(+)_R.PfoI [Ap^{r}]. DNA sequence of R.PfoI gene was determined by DNA sequencing of pET-R.PfoI plasmid isolated from two selected clones. To test the expression of R.PfoI Escherichia coli BL21 (DE3) cells carrying plasmid pJRD-PfoM6 were transformed with pET21b(+)_R.PfoI and R.PfoI expression was induced. The expression of R.PfoI was not observed under the tested experimental conditions. Therefore, another strategy was needed to express R.PfoI, because in order to express R.PfoI in E. coli it was necessary to overcome the problems associated with incomplete modification by M.PfoI.

### Example 6. Expression of pfoIR Gene in E. coli

In order to express R.PfoI in E. coli, it was necessary to overcome the problems associated with incomplete modification by M.PfoI. Non-cognate methylase was tested to determine whether protection against R.PfoI cleavage could be achieved. Ec118kI m5C methylase specific for the [symbol] sequence (modified cytosine bold/italics) was used to protect host DNA from R.PfoI cleavage since methylation activity of M.Ec118kI overlaps with possible cognate methylation of M.PfoI (rebase.neb.com/cgi-bin/msget?PfoI; Tamulaitis et al 2015).

DH10B carrying Ec118kI methyltransferase gene in plasmid pACYC_M.Ec118kI [Cm^{r}] was further used as a host for transformation of pET21b(+)_R.PfoI [Ap^{r}]. To express R.PfoI gene, plasmid DNA isolated from DH10B/pACYC_M.Ec118kI/pET-R.PfoI cells was used to transform BL21 (DE3) cells bearing pACYC_M.Ec118kI plasmid.

Cells were grown in LB medium with suitable antibiotics at 37° C. to OD600 0.7 and R.PfoI expression was induced by addition of IPTG (final concentration 1 mM). After 3.5 h of induction the cells were harvested by centrifugation and the pellet was stored at −20° C. Cell induction resulted in appearance of a 35 kDa protein band.

### Example 7. R.PfoI Activity in Cell-Free Extracts

To test the expression of R.PfoI Escherichia coli BL21 (DE3) cells (carrying plasmids pACYC_M.Ec118kI [Cmr] and pET21b(+)_R.PfoI [Apr]) were grown in the LB medium supplemented with appropriate antibiotics at 37° C. to OD600 0.7 and R.PfoI expression was induced by addition of IPTG (final concentration 1 mM). After 3.5 h incubation at 30° C. the cells were harvested by centrifugation and the pellet was stored at −20° C.

Pseudomonas fluorescens biovar 126 cells were grown in the LB3Mg medium at 30° C. to OD600 6. After 6 hours cells were harvested by centrifugation and the pellet was stored at −20° C.

To test R.PfoI activity 0.5 g of the Pseudomonas fluorescens biovar 126 biomass and 0.5 g of Escherichia coli BL21 (DE3)/pACYC_M.Ec118kI/pET21b(+)_R.PfoI biomass were re-suspended in 7 ml of Resuspension Buffer (10 mM potassium phosphate (pH 7.4), 100 mM NaCl, 1 mM EDTA, 1 mM DTT) supplemented with 2 mM phenylmethanesulfonylfluoride (PMSF) and sonicated. 10 μl of each sample were removed and used in protein electrophoresis (SDS-PAGE) analysis. The supernatants of the remaining part of the samples were obtained by centrifugation of sonicated cells at 16000 rcf for 30 min at 4° C. and were used for restriction endonuclease activity measurement.

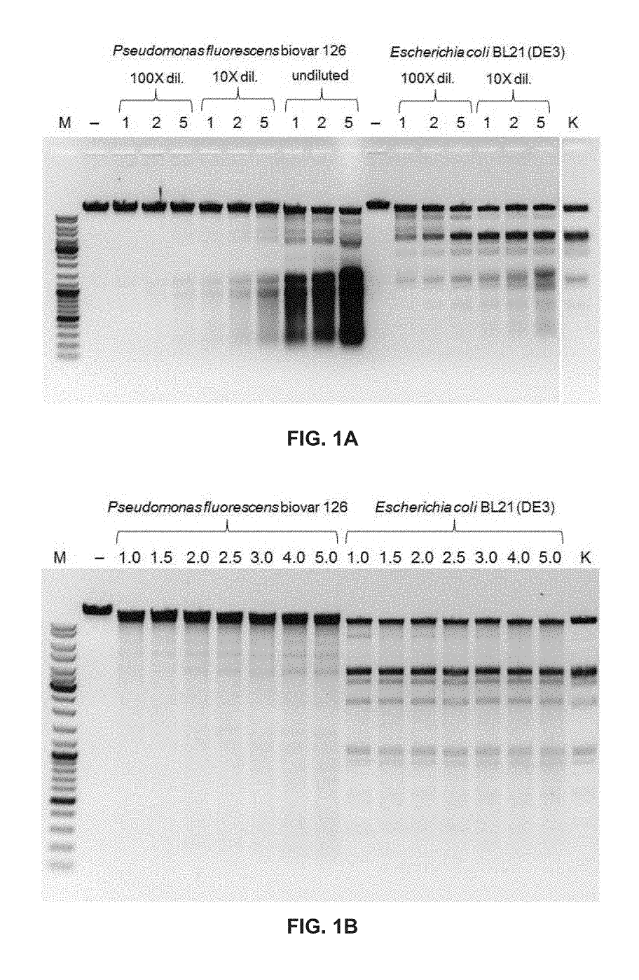

Supernatants were diluted 0, 10, 50 and 100 times in Modrich buffer (20 mM potassium phosphate (pH 7.4), 200 mM KCl, 1 mM EDTA, 7 mM 2-mercaptoethanol, 0.2 mg/ml BSA, 10% glycerol). 1, 2 and 5 μl of each undiluted, 10 times diluted and 100 times diluted extracts were incubated with 1 μg of lambda DNA (dam-dcm-) in 50 μl of 1× Tango™ Buffer (Thermo Scientific) for 10 minutes at 37° C. Reaction was stopped by adding 10 μl of 6×DNA Loading Dye & SDS (Thermo Scientific) and incubating for 10 min at 70° C. The reaction products were analyzed by agarose gel electrophoresis on a 1% agarose gel in 1×TAE buffer (FIG. 1, (A)). The results show that 100 times diluted extracts of BL21 (DE3)/pACYC_M.Ec118kI/pET21b(+)_R.PfoI already provide the level of R.PfoI activity that is available only in undiluted extract of PfoI native strain Pseudomonas fluorescens biovar 126. This suggests improved yield of recombinant PfoI restriction endonuclease compared to PfoI natively expressed in Pseudomonas fluorescens biovar 126.

1.0, 1.5, 2.0, 2.5, 3.0, 4.0 and 5.0 μl of the 50 times diluted extracts were incubated with 1 μg of lambda DNA (dam-dcm-) in 50 μl of 1× Tango™ Buffer (Thermo Scientific) for 1 hour at 37° C. Reaction was stopped by adding 10 μl of 6×DNA Loading Dye & SDS (Thermo Scientific) and incubating for 10 min at 70° C. The reaction products were analyzed by agarose gel electrophoresis on a 1% agarose gel in 1×TAE buffer (FIG. 1, (B)). The results show that in samples with extracts from Pseudomonas fluorescens biovar 126, complete digestion of lambda DNA was still not achieved after prolonged incubation.

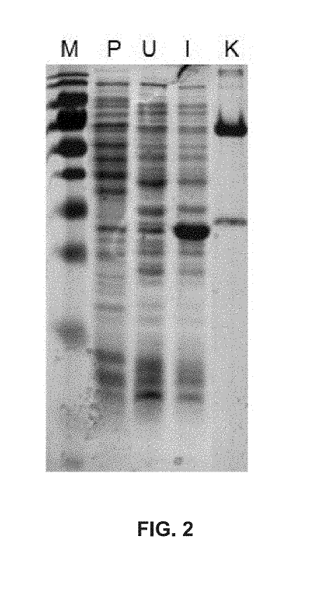

For protein electrophoresis (SDS-PAGE) analysis, 10 μl of sonicated samples were each mixed with 25 μl of 4×SDS Gel Loading Dye, 5 μl of 2 M DTT and 60 μl deionised water. For non-induced cell-extract samples (U lane in FIG. 2), 0.71 ml of BL21 (DE3)/pACYC_M.Ec118kI/pET21b(+)_R.PfoI cells were removed before induction by IPTG. The cells were centrifuged, the pellet was resuspended in 70 μl of deionised water and then mixed with 25 μl of 4×SDS Gel Loading Dye and 5 μl of 2M DTT. All samples were heated at 95° C. for 5 min, and 5 μl were loaded in a 12% SDS-PAGE gel. For control sample K, 14 μl of PfoI (10 u/μl; Cat. No. ER1751, Thermo Scientific) was mixed with 5 μl of 4×SDS Gel Loading Dye and 1 μl of 2 M DTT; the sample was heated at 95° C. for 5 min and 10 μl were loaded in the SDS-PAGE gel. Protein electrophoresis results (FIG. 2) show that E. coli BL21 (DE3)/pACYC_M.Ec118kI/pET21b(+)_R.PfoI produces significantly larger amounts of restriction endonuclease compared to the native strain Pseudomonas fluorescens biovar 126.

### Example 8. Purification of R.PfoI

To isolate R.PfoI the cells were re-suspended in purification buffer (10 mM potassium phosphate (pH 7.4), 100 mM NaCl, 1 mM EDTA, 7 mM 2-mercaptoethanol) supplemented with 2 mM phenylmethanesulfonylfluoride (PMSF) and sonicated. The supernatant was subjected to a subsequent chromatography on HiTrap™ Heparin and MonoQ 5/50 columns (GE Healthcare). Proteins were eluted by gradient of NaCl. Fractions containing PfoI endonuclease were pooled and dialysed against the storage buffer (10 mM Tris-HCl (pH 8.0 at 25° C.), 300 mM KCl, 1 mM DTT, 1 mM EDTA (ethylenediaminetetraacetic acid), 50% glycerol) and stored at −20° C. The protein preparation was >90% pure according to a Coomassie blue-stained sodium dodecyl sulphate (SDS)-gel. Concentration of the protein was determined measuring absorption at 280 nm using extinction coefficient 35143/M*cm for PfoI, as calculated by the ProtParam tool (web.expasy.org/protparam/).

### Example 9. Sequence Alignment

The amino acid sequence of R.PfoI (SEQ ID NO: 2) was used to perform a protein to protein (blastp) BLAST search, or a protein to translated database (tblastn) BLAST search. For example, such a search may be performed through the NCBI web server: ncbi.nlm.nih.gov/blast/selecting the blastp (or tblastn) program, and searching against the NR (non-redundant) database of “all organisms,” using the standard preset values, which consist of Expect=10, word size=3, using the BLOSUM62 matrix and with gap costs of Existence=11, extension=1. These parameters may also be varied by those skilled in the art to obtain slightly varied search results.

The output returned by the BLAST search is examined for sequences that give Expectation scores of less than e-02. These sequences are presumed to be restriction endonucleases (REs).

The sequence context of the putative REs identified is examined to see if there is a putative DNA methyltransferase adjacent or near (within one or two ORFS) the putative endonuclease. The presence of such a methyltransferase is highly suggestive that the sequence identified using the known endonuclease sequence is an endonuclease.

The level of similarity between R.PfoI and the newly identified sequence can suggest whether the two sequences are isoschizomers (high degree of similarity, for example E<e-50) or may recognize related but different sequences (lesser degree of similarity).

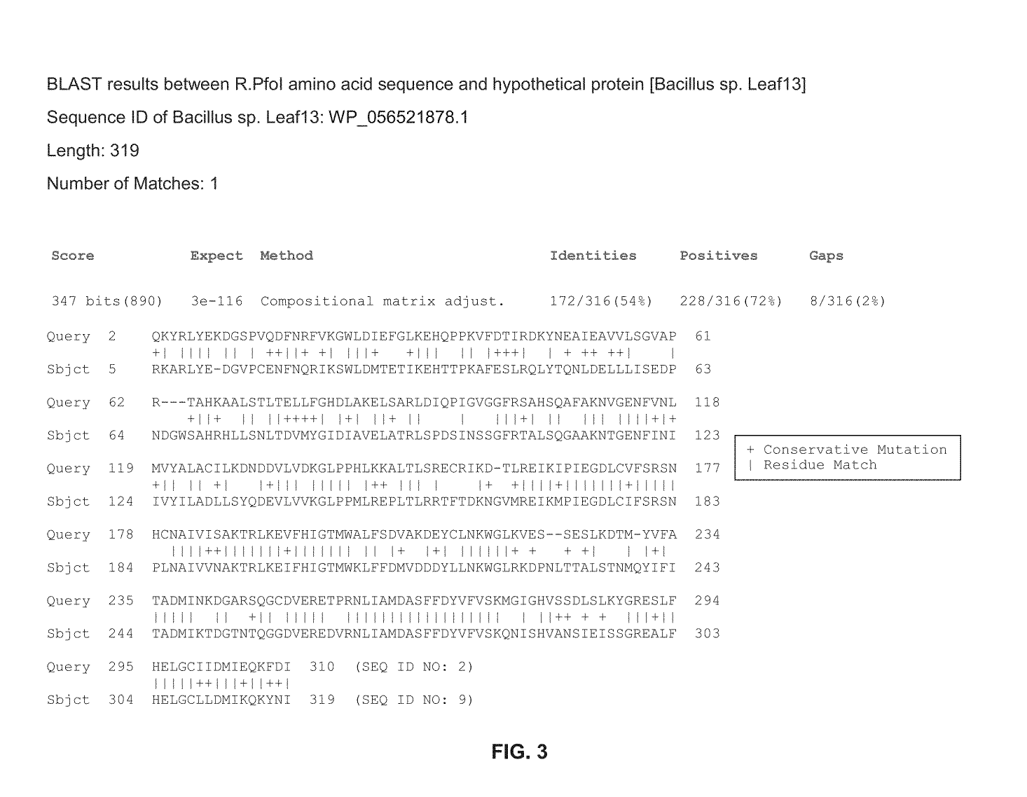

A representative BLAST search result is shown in FIG. 3. The amino acid sequence of R.PfoI shows a 54% match with hypothetical protein [Bacillus sp. Leaf13] (Accession Number: WP_056521878). These data confirm that the amino acid sequence for R.PfoI (and similarly the nucleic acid sequence encoding it) are not part of a tightly-clustering homologous group of restriction endonucleases with sequence similarity. The BLAST search shows that these sequences are quite dissimilar to other sequences.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

What is claimed is:

1. A recombinant vector comprising a nucleic acid that encodes a restriction endonuclease polypeptide with at least 90% sequence identity to SEQ ID NO: 2, wherein the restriction endonuclease cleaves DNA at the nucleotide sequence TCCNGGA.

2. A recombinant vector of claim 1, wherein the nucleic acid encodes a restriction endonuclease polypeptide of SEQ ID NO: 2.

3. The recombinant vector of claim 1, further comprising at least one regulatory element that is not from Pseudomonas fluorescens.

4. The recombinant vector of claim 2, further comprising at least one regulatory element that is not from Pseudomonas fluorescens.

5. The recombinant vector of claim 3, wherein the regulatory element comprises at least one of a promoter, a translation initiation sequence, a termination codon, a transcription termination sequence, or a sequence encoding a protein tag.

6. The recombinant vector of claim 4, wherein the regulatory element comprises at least one of a promoter, a translation initiation sequence, a termination codon, a transcription termination sequence, or a sequence encoding a protein tag.

7. A host cell transformed by a recombinant vector according to claim 1.

8. The host cell of claim 7, wherein the host cell comprises bacterial expression cells, yeast expression cells, algae expression cells, insect expression cells, mammalian expression cells, and cell-free in vitro expression systems.

9. The host cell of claim 8, wherein the host cell is E. coli.

10. A host cell transformed with a recombinant vector comprising a nucleic acid sequence of SEQ ID NO: 1, and wherein a restriction endonuclease encoded by SEQ ID NO: 1 binds to the nucleotide sequence TCCNGGA and cleaves between T and C in each strand, producing DNA fragments with protruding pentanucleic 5′-ends.

11. A method of producing recombinant PfoI restriction endonuclease from the host cell according to claim 7, the method comprising:
(i) premodifying the DNA sequence TCCNGGA in the host cell by methylation at one or more nucleotides; and
(ii) culturing the premodified host cell under conditions for expression of PfoI restriction endonuclease.

12. A method according to claim 11, wherein premodifying the DNA in the host cell is achieved by;
(i) transforming the host cell with a vector comprising a recombinant DNA of SEQ ID NO: 8; and
(ii) causing methylation of the host cell DNA by culturing the host cell under conditions for expression of M. Ec118kI.

13. A method of producing recombinant PfoI restriction endonuclease from the host cell according to claim 10, the method comprising:
(i) premodifying the DNA sequence TCCNGGA in the host cell by methylation at one or more nucleotides; and
(ii) culturing the premodified host cell under conditions for expression of PfoI restriction endonuclease.

14. A method according to claim 13, wherein premodifying the DNA in the host cell is achieved by;
(i) transforming the host cell with a vector comprising a recombinant DNA of SEQ ID NO: 8; and
(ii) causing methylation of the host cell DNA by culturing the host cell under conditions for expression of M. Ec118kI.

15. A method of producing an endonuclease, comprising culturing the host cell transformed with a recombinant vector comprising a nucleic acid sequence of SEQ ID NO: 1 under conditions suitable for expression of the endonuclease, wherein a restriction endonuclease encoded by SEQ ID NO: 1 cleaves DNA at the nucleotide sequence TCCNGGA.

16. The method of claim 15, wherein the host cell is also transformed with a vector comprising a recombinant DNA encoding a methyltransferase capable to modify one or more nucleotides of the host cell DNA sequence TCCNGGA.

17. The method of claim 11, wherein the vector comprises a recombinant DNA of SEQ ID NO: 8.

18. A method of producing an endonuclease, comprising culturing the host cell of claim 7 under conditions suitable for expression of the endonuclease.

19. The method of claim 17, wherein the host cell is also transformed with a vector comprising a recombinant DNA encoding a methyltransferase capable to modify one or more nucleotides of the host cell DNA sequence TCCNGGA.

20. The method of claim 11, wherein the vector comprises a recombinant DNA of SEQ ID NO: 8.


SEQUENCE LISTING (9 sequences)

SEQ ID NO: 1 - DNA, 936 bp, Artificial Sequence
  R.PfoI nucleic acid sequence
atgcagaagtataggttgtatgagaaggatggctcgcctgttcaagacttcaatcgatttgtaaaaggctggttagatattgagtttggtttaaaggagcatcagccgccaaaggtatttgatactataagagataagtataacgaagctatagaagctgttgtattgagcggtgttgcgccgcggacggcacataaggctgcgctttccactttgaccgaacttctatttggtcatgatcttgcaaaagaacttagtgctcgtttggatattcagcccataggagtaggcggatttcgttcggcgcattctcaagcatttgctaaaaatgttggcgagaactttgtaaatttaatggtctatgcgcttgcgtgtatcttgaaagataatgatgatgttctggtagataaggggctgccacctcatctaaaaaaagctctaacattgagtcgtgagtgtaggattaaagatacgcttcgtgaaataaagatccctatagaaggggatctgtgtgttttttcacgaagtaatcattgcaatgcaatcgttattagcgctaaaacaagactcaaagaagtctttcatattggcacaatgtgggcgttgttttctgatgtggcgaaagatgaatactgtcttaacaagtgggggctgaaagttgagtcgtcagagtccctcaaggatacgatgtatgtatttgctacagctgatatgataaataaagatggtgcaagaagtcagggttgtgatgtggagagggaaactccaagaaacttaattgcgatggacgcctctttttttgattatgtttttgtttcgaagatgggtatcgggcatgtctcttccgatctttctcttaagtatgggcgagaatcgctcttccacgagctagggtgcataattgatatgatcgaacaaaagtttgatattttactt

SEQ ID NO: 2 - protein, 312 aa, Artificial Sequence
  R.PfoI amino acid sequence
MQKYRLYEKDGSPVQDFNRFVKGWLDIEFGLKEHQPPKVFDTIRDKYNEAIEAVVLSGVAPRTAHKAALSTLTELLFGHDLAKELSARLDIQPIGVGGFRSAHSQAFAKNVGENFVNLMVYALACILKDNDDVLVDKGLPPHLKKALTLSRECRIKDTLREIKIPIEGDLCVFSRSNHCNAIVISAKTRLKEVFHIGTMWALFSDVAKDEYCLNKWGLKVESSESLKDTMYVFATADMINKDGARSQGCDVERETPRNLIAMDASFFDYVFVSKMGIGHVSSDLSLKYGRESLFHELGCIIDMIEQKFDILL

SEQ ID NO: 3 - DNA, 6349 bp, Artificial Sequence
  pET21-R.PfoI construct
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgcagaagtataggttgtatgagaaggatggctcgcctgttcaagacttcaatcgatttgtaaaaggctggttagatattgagtttggtttaaaggagcatcagccgccaaaggtatttgatactataagagataagtataacgaagctatagaagctgttgtattgagcggtgttgcgccgcggacggcacataaggctgcgctttccactttgaccgaacttctatttggtcatgatcttgcaaaagaacttagtgctcgtttggatattcagcccataggagtaggcggatttcgttcggcgcattctcaagcatttgctaaaaatgttggcgagaactttgtaaatttaatggtctatgcgcttgcgtgtatcttgaaagataatgatgatgttctggtagataaggggctgccacctcatctaaaaaaagctctaacattgagtcgtgagtgtaggattaaagatacgcttcgtgaaataaagatccctatagaaggggatctgtgtgttttttcacgaagtaatcattgcaatgcaatcgttattagcgctaaaacaagactcaaagaagtctttcatattggcacaatgtgggcgttgttttctgatgtggcgaaagatgaatactgtcttaacaagtgggggctgaaagttgagtcgtcagagtccctcaaggatacgatgtatgtatttgctacagctgatatgataaataaagatggtgcaagaagtcagggttgtgatgtggagagggaaactccaagaaacttaattgcgatggacgcctctttttttgattatgtttttgtttcgaagatgggtatcgggcatgtctcttccgatctttctcttaagtatgggcgagaatcgctcttccacgagctagggtgcataattgatatgatcgaacaaaagtttgatattttactttaaaatcacttttcctccgtcctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 4 - DNA, 22 bp, Artificial Sequence
  Primer for PCR
atcccagcaccattcacaaccc

SEQ ID NO: 5 - DNA, 22 bp, Artificial Sequence
  Primer for PCR
gcatccactacgcccgctgcac

SEQ ID NO: 6 - DNA, 30 bp, Artificial Sequence
  Primer for amplification
gagggcatatgcagaagtataggttgtatg

SEQ ID NO: 7 - DNA, 26 bp, Artificial Sequence
  Primer for amplification
gaaaagtgattttaaagtaaaatatc

SEQ ID NO: 8 - DNA, 1137 bp, Artificial Sequence
  M.Ecl18kI nucleic acid sequence
atgactgataatatcgcagcaacaatcaaagaaaaaagagaaagacttcatatgactcaaaaagagtttgcagacgcccttggtctttcaaaatatggagacagaaccataagaagatgggagcgcggggaaacaaaacctactggggcggaactaaaagcagttatggactttccagacacaccaccttacccaaacaacgagaatggcagatatcgcatgatcgatttatttgctggaatcggtggaaccagacttggatttcatcaaactaacgctgtaaatgttgttttttccagcgaatgggataagtttgctcaaaagacatatcatgcaaattatggtgatttccctgatggggacataacaaaaatcgatgaaaaagatattcctgatcatgaaattcttgtcggtgggtttccttgcgtagcattctctcaagcagggctcaaaaaaggatttaatgatacaagaggaactttgttttttgatatagcaaggataataaaggaaaaaaaacctcatgcttttttacttgaaaatgtaaaaaacttgctagggcatgataaaggtagaactttttcaataataaaaaatacacttgaagaattgaattacactgtgtattacaatatcttcgctgcaaaagactttggtgttccacaaaatagagagcgcatatacattgtaggctttaataaagaaaaagtaagaaatcatgaacattttacttttccaaccccattaaaaacaaaaacaagagttggagatattcttgaaaagagcgtagacaataaatatacgctatcagatgcgctctggaatggacaccaacgcaggaaacttgtaaatgcagcagccggtaaaggctttgggtatggtttatttaatgaaaatagcccatatactaataccataagtgcgaggtactataaagatggtagcgaaatattaatagagcaaaaaggaagtaacccaagaaaaataactccgcgggaagcgtcaagacttcaagggtttcctagtgattttattatccctgtatcggatacacaggcttataagcaatttgggaattcagtcgcagtccctgttatcaatgcaatagcagaaaaaatcatatcgaccttagattca

SEQ ID NO: 9 - protein, 319 aa, Artificial Sequence
  Amino acid sequence of hypothetical protein [Bacillus sp. Leaf13]
MAVRRKARLYEDGVPCENFNQRIKSWLDMTETIKEHTTPKAFESLRQLYTQNLDELLLISEDPNDGWSAHRHLLSNLTDVMYGIDIAVELATRLSPDSINSSGFRTALSQGAAKNTGENFINIIVYILADLLSYQDEVLVVKGLPPMLREPLTLRRTFTDKNGVMREIKMPIEGDLCIFSRSNPLNAIVVNAKTRLKEIFHIGTMWKLFFDMVDDDYLLNKWGLRKDPNLTTALSTNMQYIFITADMIKTDGTNTQGGDVEREDVRNLIAMDASFFDYVFVSKQNISHVANSIEISSGREALFHELGCLLDMIKQKYNI